(19) United States
(12) Patent Application Publication
(10) Pub. No.: US 2011/0269992 A1
(43) Pub. Date: Nov. 3, 2011

(54) Preparation of acetic acid and acetic anhydride

(76) Inventors: Shao-Hua Guo, Exton, PA (US); Wayne J. Brtko, Glen Mills, PA (US); Noel Hallinan, Loveland, OH (US); Brian A. Salisbury, Oxford, PA (US)

(21) Appl. No.: 12/799,663

(22) Filed: Apr. 29, 2010

(51) Int. Cl. C07C 51/12

(52) U.S. Cl. 562/517

(57) ABSTRACT

Disclosed is a process for the preparation of acetic acid and acetic anhydride. The process comprises carbonylating dimethyl carbonate. The carbonylation reaction for producing acetic acid is performed in the presence of water, while the carbonylation for producing acetic anhydride is performed essentially in the absence of water.

### FIELD OF THE INVENTION

The invention relates to the preparation of acetic acid and acetic anhydride. More particularly, the invention relates to the preparation of acetic acid and acetic anhydride from dimethyl carbonate.

### BACKGROUND OF THE INVENTION

The carbonylation of methanol produces acetic acid. A rhodium catalyst for methanol carbonylation was developed by Monsanto. The rhodium catalyst allows low reaction temperature and pressure and gives high selectivity to acetic acid. Celanese modified the Monsanto process by adding lithium iodide salt to the carbonylation. Lithium iodide increases the catalyst stability in a low water carbonylation process. Lyondell Chemical Company developed a new rhodium carbonylation catalyst system that uses a pentavalent Group VA oxide such as triphenylphosphine oxide as a catalyst stabilizer. The Lyondell catalyst system not only reduces the amount of water needed to stabilize the catalyst but also increases the carbonylation rate and acetic acid yield. See U.S. Pat. No. 5,817,869.

Acetic anhydride can be made by dehydration of acetic acid. It is, however, commercially manufactured by two other processes. The first process is the so called ketene-acetic acid technology, which involves thermal cracking acetic acid to ketene and subsequently reacting ketene with additional acetic acid to form acetic anhydride. The second process is methyl acetate carbonylation. Suitable catalysts for the carbonylation of methyl acetate are essentially the same as those used in the carbonylation of methanol to acetic acid. See U.S. Pat. No. 4,046,807. Acetic anhydride is mainly consumed in manufacturing cellulose acetate esters.

Dimethyl carbonate (DMC) is known. DMC is mainly used in manufacturing polycarbonate resins and lithium batteries. It has been increasingly used in coatings, inks, and adhesives and has been exempted from the definition of volatile organic compounds (VOC) by the U.S. EPA. There are many ways to make DMC, including transesterification of propylene carbonate or ethylene carbonate with methanol, reaction of methanol with urea, oxidative carbonylation of methanol, and reaction of phosgene with methanol.

New processes for producing acetic acid and acetic anhydride are needed. Ideally, the process produces acetic anhydride without using acetic acid as a starting material.

### SUMMARY OF THE INVENTION

The process of the invention comprises carbonylating dimethyl carbonate to acetic acid and acetic anhydride. The carbonylation reaction for producing acetic acid is performed in the presence of water, and the carbonylation for producing acetic anhydride is performed essentially in the absence of water. Preferably, the carbonylation is performed in the presence of a rhodium catalyst and a catalyst stabilizer selected from pentavalent Group VA oxides.

### DETAILED DESCRIPTION OF THE INVENTION

The carbonylation of dimethyl carbonate is performed in the presence of a carbonylation catalyst. Suitable carbonylation catalysts include those known to the industry. Examples of suitable carbonylation catalysts include rhodium catalysts and iridium catalysts.

Suitable rhodium catalysts are taught, for example, by U.S. Pat. No. 5,817,869. Suitable rhodium catalysts include rhodium metal and rhodium compounds. Preferably, the rhodium compounds are selected from the group consisting of rhodium salts, rhodium oxides, rhodium acetates, organo-rhodium compounds, coordination compounds of rhodium, the like, and mixtures thereof. More preferably, the rhodium compounds are selected from the group consisting of Rh₂(CO)₄I₂, Rh₂(CO)₄Br₂, Rh₂(CO)₄Cl₂, Rh(CH₃CO₂)₂, Rh(CH₃CO₂)₃, [H]Rh(CO)₂I₂, the like, and mixtures thereof. Most preferably, the rhodium compounds are selected from the group consisting of [H]Rh(CO)₂I₂, Rh(CH₃CO₂)₃, the like, and mixtures thereof.

Suitable iridium catalysts are taught, for example, by U.S. Pat. No. 5,932,764. Suitable iridium catalysts include iridium metal and iridium compounds. Examples of suitable iridium compounds include IrCl₃, IrI₃, IrBr₃, [Ir(CO)₂I]₂, [Ir(CO)₂Cl]₂, [Ir(CO)₂Br]₂, [Ir(CO)₄I₂]⁻H⁺, [Ir(CO)₂Br₂]⁻H⁺, [Ir(CO)₂I₂]⁻H⁺, [Ir(CH₃)I₃(CO)₂]⁻H⁺, Ir₄(CO)₁₂, IrCl₃4H₂O, IrBr₃4H₂O, Ir₃(CO)₁₂, Ir₂O₃, IrO₂, Ir(acac)(CO)₂, Ir(acac)₃, Ir(OAc)₃, [Ir₃O(OAc)₆(H₂O)₃][OAc], and H₂[IrCl₆]. Preferably, the iridium compounds are selected from the group consisting of acetates, oxalates, acetoacetates, the like, and mixtures thereof. More preferably, the iridium compounds are acetates.

The iridium catalyst is preferably used with a co-catalyst. Preferred co-catalysts include metals and metal compounds selected from the group consisting of osmium, rhenium, ruthenium, cadmium, mercury, zinc, gallium, indium, and tungsten, their compounds, the like, and mixtures thereof. More preferred co-catalysts are selected from the group consisting of ruthenium compounds and osmium compounds. Most preferred co-catalysts are ruthenium compounds. Preferably, the co-catalysts are chloride-free such as acetates.

The carbonylation catalyst is used in an amount preferably within the range of 0.0001 to 10%, and more preferably within the range of 0.001 to 1%, of the total weight of the reaction medium.

Preferably, the carbonylation is performed in the presence of a catalyst promoter. Preferably, the catalyst promoter is an iodide. More preferably, the catalyst promoter is an alkyl iodide. Most preferably, the catalyst promoter is methyl iodide. Preferably, the concentration of methyl iodide is from about 0.6 wt % to about 36 wt % based on the total weight of the reaction medium. More preferably, the concentration of methyl iodide is from about 4 wt % to about 24 wt %. Most preferably, the concentration of methyl iodide is from about 6 wt % to about 20 wt %.

Preferably, the reaction is performed in the presence of a catalyst stabilizer. Suitable catalyst stabilizers include those known to the industry. In general, there are two types of catalyst stabilizers. The first type of catalyst stabilizer is metal iodide salt such as lithium iodide. The second type of catalyst stabilizer is a non-salt stabilizer. Preferred non-salt stabilizers are pentavalent Group VA oxides. See U.S. Pat. No. 5,817,869. Phosphine oxides are more preferred. Triphenylphosphine oxides are most preferred. The catalyst stabilizer is present in a molar ratio of stabilizer/catalyst preferably within a range of 1:1 to 5000:1, more preferably within the range of 60:1 to 5000:1, and most preferably within the range of 60:1 to 500:1.

Optionally other methyl sources such as methyl acetate and methyl ether can be added to the carbonylation reaction. Methyl acetate or methyl ether can be added to the carbonylation reaction in any ratio to dimethyl carbonate. Preferably, the weight ratio of methyl acetate or methyl ether to dimethyl carbonate is within the range of 1:99 to 99:1, more preferably within the range of 5:95 to 95:5, and most preferably within the range of 10:90 to 90:10.

Hydrogen may also be fed into the reactor. Addition of hydrogen can enhance the carbonylation efficiency. Preferably, the concentration of hydrogen is from about 0.1 mol % to about 5 mol % of carbon monoxide in the reactor. More preferably, the concentration of hydrogen is from about 0.3 mol % to about 3 mol % of carbon monoxide in the reactor.

Carbon monoxide can be fed to the carbonylation reactor in a separate stream or it can be premixed with dimethyl carbonate or other starting materials such as hydrogen. It is preferably used in a molar ratio of carbon monoxide/dimethyl carbonate within the range of 1:1 to 10:1.

The carbonylation reaction is preferably performed at a temperature within the range of about 150° C. to about 250° C. More preferably, the reaction is performed at a temperature within the range of about 150° C. to about 200° C. The carbonylation reaction is preferably performed under a pressure within the range of about 200 psig to about 2,000 psig. More preferably, the reaction is performed under a pressure within the range of about 300 psig to about 500 psig.

The carbonylation reaction for producing acetic anhydride is performed essentially in the absence of water.

The carbonylation reaction for producing acetic acid is performed in the presence of water. Preferably, the concentration of water present is from about 2 wt % to about 14 wt % based on the total weight of the reaction medium. More preferably, the water concentration is from about 2 wt % to about 10 wt %. Most preferably, the water concentration is from about 4 wt% to about 8 wt %. Depending on the water concentration, a mixture of acetic acid and acetic anhydride can be co-produced if it is desirable.

Optionally, methanol can be added to the carbonylation reaction for producing acetic acid. Methanol can be added to the carbonylation reaction in any ratio to dimethyl carbonate. Preferably, the weight ratio of methanol to dimethyl carbonate is within the range of 1:99 to 99:1, more preferably within the range of 5:95 to 95:5, and most preferably within the range of 10:90 to 90:10.

The following example merely illustrates the invention. Those skilled in the art will recognize many variations that are within the spirit of the invention and scope of the claims.

### EXAMPLE 1

### Production of Acetic Acid

To a 300 mL autoclave, outfitted with a magnetically driven stirrer, is added a solution (200 mL) that contains 0.5 M hydriodic acid, 3.3 M water, 1.0 M triphenylphosphine oxide, and 0.35 M dimethyl carbonate in acetic acid. This reactor contents are heated to 185° C. and pressured by 100 psig of carbon monoxide. A solution (10 mL) that contains 0.04 M rhodium triacetate in a mixture of 9:1 acetic acid: water is injected into the autoclave via differential pressure. Immediately following the catalyst injection the reactor pressure is raised to and held at 400 psig by continuously feeding carbon monoxide. The reaction is allowed to proceed for about 1 hour. The rate of the acetic acid production is determined by monitoring the carbon monoxide uptake and converting that data into moles of carbon monoxide consumed (see Table 1). Table 1 indicates that the reaction remains essentially constant after 30 to 40 minutes.

TABLE 1
Consumption of Carbon Monoxide vs. Reaction Time

| Reaction | CO Consumption |
|---|---|
| Time (min) | (mole) |
| 0 | 0 |
| 2 | 0.017 |
| 4 | 0.032 |
| 6 | 0.047 |
| 8 | 0.061 |
| 10 | 0.074 |
| 20 | 0.110 |
| 30 | 0.123 |
| 40 | 0.130 |
| 45 | 0.133 |

We claim:

1. A process for producing acetic acid comprising carbonylating dimethyl carbonate in the presence of a rhodium catalyst, a catalyst stabilizer selected from pentavalent Group VA oxides, and water.

2. The process of claim 1, wherein the carbonylation is performed in the presence of methyl iodide.

3. The process of claim 1, wherein the carbonylation is performed in the presence of methanol.

4. The process of claim 1, wherein the catalyst stabilizer is triphenylphosphine oxide.

5. A process of making acetic anhydride comprising carbonylating dimethyl carbonate in the presence of a rhodium catalyst, a catalyst stabilizer selected from pentavalent Group VA oxides, and essentially in absence of water.

6. The process of claim 5, wherein the carbonylation is performed in the presence of methyl iodide.

7. The process of claim 5, wherein the catalyst stabilizer is triphenylphosphine oxide.
